(19) United States
(12) Patent Application Publication
(10) Pub. No.: US 2024/0101507 A1
(43) Pub. Date: Mar. 28, 2024

(54) INTEGRATED PROCESS FOR MAKING DIASTEREOPURE ETHYLENEDIAMINE DISUCCINIC ACID (EDDS) FROM ETHYLENE AND ASPARTIC ACID USING BROMINE AS AN INTERMEDIATE

(71) Applicant: SPERO RENEWABLES, LLC, Goleta, CA (US)

(72) Inventors: Ian M. KLEIN, Goleta, CA (US); Daniel MORGAN, Goleta, CA (US); Eric MCFARLAND, Goleta, CA (US); Mahdi M. ABU-OMAR, Goleta, CA (US)

(21) Appl. No.: 18/264,167

(22) PCT Filed: Feb. 3, 2022
(86) PCT No.: PCT/US2022/015037

§ 371 Date: Aug. 3, 2023

Related U.S. Application Data
(60) Provisional application No. 63/145,175, filed on Feb. 3, 2021.

(51) Int. Cl. C07C 227/18

(52) U.S. Cl. CPC C07C 227/18

(57) ABSTRACT

A process for synthesizing (S,S) ethylenediamine-N,N′-disuccinic acid (EDDS) includes combing an aqueous solution of aspartic acid with dibromoethane under basic conditions to form a reaction solution; heating the reaction solution to form a heated reaction solution, and recovering at least a portion of the reaction product from the reaction solution. The heated reaction solution comprises a reaction product, wherein the reaction product comprises (S,S) ethylenediamine-N,N′-disuccinic acid (EDDS).

### CROSS-REFERENCE TO RELATED APPLICATIONS

This application claims priority to U.S. Provisional Patent Application No. 63/145,175 filed on Feb. 3, 2021, and entitled “INTEGRATED PROCESS FOR MAKING DISTEREOPURE ETHYLENEDIAMINE DISUCCINIC ACID (EDDS) FROM ETHYLENE AND ASPARTIC ACID USING BROMINE AS A REGENERABLE INTERMEDIATE,” which is incorporated herein in its entirety by reference.

### STATEMENT REGARDING FEDERALLY SPONSORED RESEARCH OR DEVELOPMENT

None.

### BACKGROUND

EDTA is one of the most ubiquitous chelating ligands on the market due to its strong metal binding and can be found in many products from food to shampoo. Ethylenediamine-N,N′-disuccinic acid (EDDS) has similar metal complexation constants to those of EDTA, except the alkaline Earth metals where EDTA has greater complexation constants than EDDS. In general, EDDS has been sought out by manufacturers as an eco-friendly alternative to EDTA; however, the high price of EDDS prohibited its use in most applications.

### SUMMARY

In some embodiments, a process for synthesizing (S,S) ethylenediamine-N,N′-disuccinic acid (EDDS) comprises combining an aqueous solution of aspartic acid with dibromoethane under basic conditions to form a reaction solution; heating the reaction solution to form a heated reaction solution, and recovering at least a portion of the reaction product from the reaction solution. The heated reaction solution comprises a reaction product, wherein the reaction product comprises (S,S) ethylenediamine-N,N′-disuccinic acid (EDDS).

In some embodiments, a process for synthesizing (S,S) EDDS from L-aspartic acid and dibromoethane comprises combining an aqueous solution of L-aspartic acid with dibromoethane under basic conditions to form a reaction solution, heating the reaction solution to form a heated reaction solution, cooling the heated reaction solution to form a cooled reaction solution, precipitating at least a portion of the reaction product from the cooled reaction solution, and recovering the portion of the reaction product from the cooled reaction solution, wherein the reaction product comprises ≥40% pure (S,S)-EDDS. The heated reaction solution comprises a reaction product, wherein the reaction product comprises (S,S) ethylenediamine-N,N′-disuccinic acid (EDDS).

In some embodiments, a process for synthesizing (S,S)-EDDS from L-aspartic acid and dibromoethane comprises combining an aqueous solution of L-aspartic acid with dibromoethane under basic conditions to form a reaction solution, forming a reaction product based on the combining, and separating the EDDS as a solid product from the reaction mixture after the forming. The reaction product comprises (S,S) ethylenediamine-N,N′-disuccinic acid (EDDS).

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description:

FIG. 1 is a schematic illustration of the structure of (2S,2′S)-2,2′-(ethane-1,2-diylbis(azanediyl))di succinic acid, (S,S)-EDDS.

FIG. 2 is a schematic illustration of an embodiment of an integrated process for synthesis of (S,S)-EDDS from ethylene and L-aspartic acid.

FIG. 3 is a schematic illustration of the chemical conversion basis for (S,S)-EDDS synthesis using bromine as an oxidizing carrier.

FIG. 4 illustrates the results of an NMR analysis of a reaction product produced from an embodiment of the process described herein.

FIG. 5 illustrates the UV-Visible Spectrum of Br2 produced from reaction of spent bromide and sodium hypochlortite.

### DETAILED DESCRIPTION

Ethylenediamine-N,N′-disuccinic acid (EDDS) is both a powerful chelating agent and also biodegradable. Disclosed herein is an integrated process for preparation of high purity EDDS, production of a halide such as bromine from a spent halide (e.g., bromide), and generation of dibromoethane from in situ generated bromine and ethylene. The dibromoethane intermediate is used for EDDS synthesis from aspartic acid and the bromine is recovered. The described innovation enables the synthesis of EDDS from ethylene and aspartic acid with stoichiometric oxidant such as bleach or chlorine using bromine as a carrier.

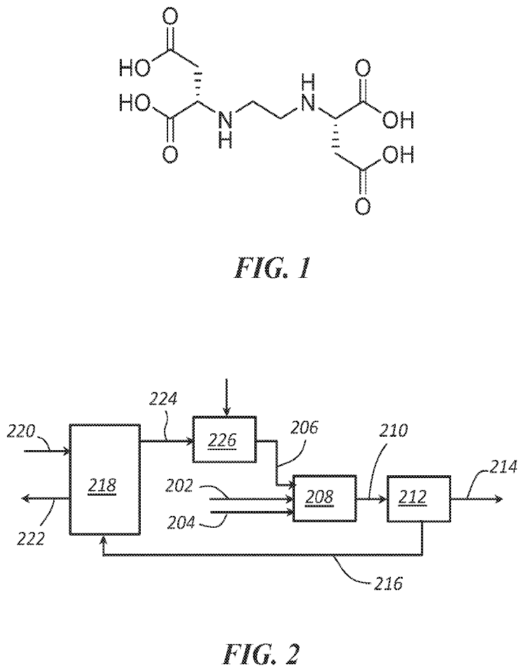

This disclosure describes the integrated process for the synthesis of (S,S) ethylenediamine-N,N′-disuccinic acid (EDDS) (where the structure is shown in FIG. 1) from aspartic acid and dibromoethane, production of bromine gas from spent bromide salts contained within the EDDS filtrate, and reaction of the bromine gas with ethylene to produce dibromoethane in situ. This process regenerates the dibromoethane using a novel method for in situ bromine recycling, where bromine generated from spent bromide in the EDDS reaction mixture is converted to bromine gas and reacted with ethylene to produce dibromoethane

(S,S) Ethylenediamine-N,N′-disuccinic acid (EDDS) is a powerful, biodegradable chelating agent used in agricultural, consumer, and industrial applications. EDDS can be synthesized from the reaction of ethylene diamine and maleic acid or from aspartic acid and dibromoethane (DBE) or dichloroethane (DCE). EDDS is a chelating agent can be produced with aspartic acid as the starting reagent. EDDS can exist as three isomers, the (S,S)-EDDS isomer is biodegradable while (R,R)-EDDS and meso-EDDS were found to be only partially or wholly non-degradable. EDDS is an advantageous chelating molecule as S,S diastereomer is readily biodegradable because it is based on the naturally occurring amino acid L-aspartic acid. In the soil, (S,S)-EDDS degrades in 7-11 days with a half-life of approximately 4.5 days. While (S,S)-EDDS is readily biodegradable, the chelating molecule ethylenediaminetetraacetic acid (EDTA) is not.

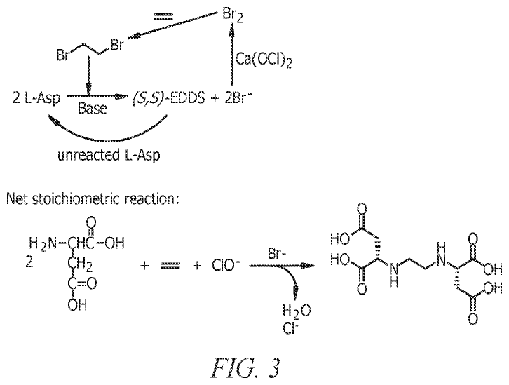

As described in the present disclosure, (S,S)-EDDS has now been synthesized at greater than 70%, greater than 80%, or greater than 90% (e.g., up to about 91% or up to about 92%) purity from aspartic acid and dibromoethane. EDDS can be produced from L-aspartic acid and dibromoethane under basic conditions as described herein with respect to FIG. 2. The aspartic acid can be first dissolved in an aqueous solution to form an aspartic acid solution in stream 202. The aqueous solution can have a basic pH of between about 8 and about 12, or between about 9 and about 11, or in some aspects, about 10. In some aspects, the pH of the aspartic acid solution can be adjusted after the dissolution of all of the aspartic acid. For example, a stream 204 of a base can be combined with the aspartic acid solution in a reactor 208 to adjust the pH of the aspartic acid solution. Any suitable base can be used such as a hydroxide (e.g., NaOH, KOH, etc.) or the like.

The aspartic acid solution can be heated to between about 50-100° C. For example, the aspartic acid solution can be heated in the reactor 208. The solution can be stirred or agitated while heating in some aspects. For example, the reactor 208 can comprise one or more mixers or agitators to mix the solution within the reactor 208.

Dibromoethane (DBE) in stream 206 can be added to the aspartic acid solution. For example, the DBE can be added to the aspartic acid solution in the reactor 208. The DBE can be added as a liquid, though in some aspects, the DBE can be added as a gas phase or partial vapor phase depending on the temperature of the reactor 208. The DBE can be added to the aspartic acid solution over time, for example, over a time between about 30 min to about 5 hours. In some aspects, the DBE can be added as a single dose such that the DBE is added at essentially the same time, which can be followed by mixing and reaction of the reaction mixture.

Following the DBE addition, the reaction temperature in the reactor 208 can be maintained at between about 50-100° C. and held for a reaction period. The reaction period can be between about 30 minutes to about 10 hours, or as an example 1 to 3 hours. The reaction pH can be maintained in the basic range with the addition of a base in stream 204 during the reaction. In some aspects, the reaction pH can be maintained at pH 9-10 by periodic addition of base. Various sensors such as a pH meter may be used to control the pH of the reaction mixture during the reaction in order to limit the production of potential by-products.

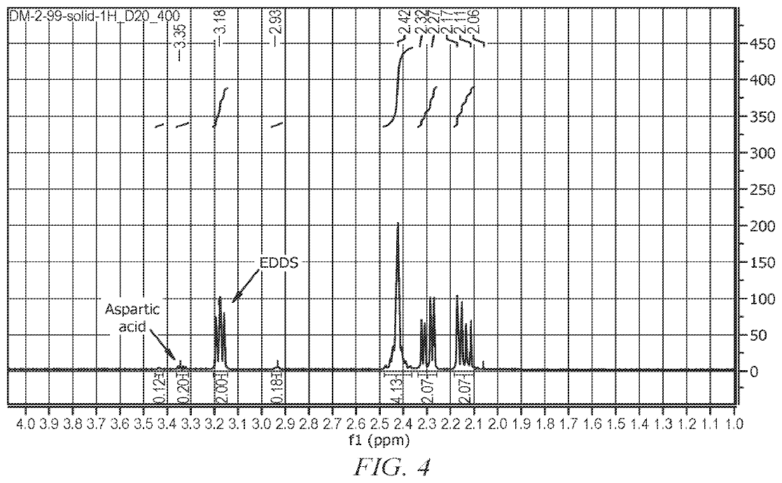

While not wishing to be limited by theory, the expected reaction scheme is shown in FIG. 3. The resulting product of the reaction of DBE with aspartic acid to produce (S,S)-EDDS is shown using bromine as an oxidizing carrier. When completed, the reaction mixture can be transferred as stream 210 to a separator 212 and cooled. While shown as a separate unit as separator 212, in some aspects, a batch or semi-batch process can be used where the reactor 208 also serves as the separator 212. The reaction mixture in the separator can be cooled to a temperature of between about 0° C. to about 40° C., or about 25° C. (e.g., about room temperature). The pH of the reaction mixture in the separator 212 can be adjusted into the acid range. For example, the reaction mixture can be acidified to a pH in the range of about 1 to about 6, between about 2 to about 4, or in a range of about 2.8-3.0 by addition of suitable acid. Any suitable acid such as a mineral acid can be used. In some aspects, hydrochloric acid (HCl) can be used to acidify the reaction mixture. The change in pH can causes the precipitation of a white solid, the majority of which can be (S,S)-EDDS, and the solid can be removed from the separator 212 as the EDDS product. In some aspects, the white solid can have a purity of greater than 40%, greater than 50%, greater than 60%, greater than 70%, greater than 80%, or greater than 90% EDDS. For example as shown in FIG. 4, Nuclear Magnetic Resonance spectroscopy (NMR) of the resulting precipitate product shows a 92% purity of (S,S)-EDDS, with the remaining 8% being L-aspartic acid.

In some aspects, the aspartic acid reaction solution may have an excess of aspartic acid. The resulting precipitate may then have a lower purity due to the presence of the precipitated excess aspartic acid. In some aspects, an additional purification step can be performed on the precipitate to further purify the EDDS product. For example, an additional dissolution and controlled precipitation can be performed in order to produce a higher purity EDDS product. Other suitable separations of the aspartic acid/EDDS mixture can be carried out to separate the aspartic acid from the EDDS to produce EDDS at a desired purity.

The liquid filtrate from the precipitation to recover the product stream can optionally be further acidified by addition of an acid (e.g., HCl, etc.) to cause a second precipitation, which can result in unreacted aspartic acid being recovered as a solid. As described in the examples, the mass balance as a result of EDDS and recovered L-aspartic acid accounts for the initial L-aspartic acid used in the reaction. Recovering the aspartic acid in large quantities may allow for its recycle and reuse in the EDDS synthesis.

The filtrate aqueous mother liquor containing spent bromide ions can pass as stream 216 to a recovery unit 218 to recover and produce bromine gas. Within the mother liquor, the bromine may be present as a salt such as NaBr, which can pass to the recovery unit 218. Within the recovery unit 218, the bromide salt in the aqueous mother liquor can be contacted with an oxidizing agent such as sodium hypochlorite (NaClO) to produce bromine gas and the corresponding salt such as sodium chloride. The pH in the recovery unit can be maintained by the addition of a suitable acid, which may correspond to the oxidizing agent and/or the desired by-product salt. For example, HCl may be used to control the pH when NaClO is used as the oxidizing agent so that sodium chloride is produced as the by-product salt. The salt solution can be removed from the recovery unit as stream 222 for further separation and handling of the salts. The gaseous bromine can pass out of the recovery unit 218 as a gas in stream 224.

The bromine gas produced in the recovery unit 218 can pass as stream 224 to the bromination unit 226. Within the bromination unit 226, the gaseous bromine can be contacted with ethylene to generate dibromoethane, which can be carried out as a gas phase reaction. The resulting DBE can pass as stream 206 to contact the aspartic acid solution in the reactor 208 to complete the reaction loop. In this reaction process, the brome acts as an intermediate that is recovered and recycled within the overall reaction. This process utilizes bromine as a carrier providing both an economic and environmental advantage for making (S,S)-EDDS from ethylene, an oxidant (e.g., sodium hypochlorite, hydrogen peroxide, molecular chlorine, molecular oxygen, other oxidants, etc.), and L-aspartic acid.

The novelty and essence of the process described herein is the generation of the expensive reactant dibromoethane from added low cost ethylene and bromine generated from the produced bromide salts in an integrated process that may be operated as a continuous, semi-continuous, or batch process with the isolation of the EDDS product at a relatively high initial purity. In some aspects, hypochlorite can be used with HCl to produce bromine in water; however, other embodiments can make use of other aqueous oxidants including hydrogen peroxide, molecular chlorine, and molecular oxygen to name a few. Gas phase bromine can be recovered from the aqueous solution of bromine without need for further drying because the subsequent reaction with ethylene is not sensitive to trace water vapor. The pH of the reaction can be controlled to avoid undesired aqueous byproducts.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

In a first example, 40 grams of aspartic acid, 300 mL of H₂O, and 15 g of NaOH were added to a 1 L round bottom flask fitted with a condenser. The solution was heated to 80° C. with stirring. Dibromoethane was added dropwise to the reaction flask with an addition funnel over 30 min to 5 hours. Additional amounts of NaOH were added to the reaction flask intermittently to maintain a pH of 9-10. The reaction mixture was maintained at 80° C. under continuous stirring for an additional 4 hours after completion of the DBE addition. The reaction mixture was removed from the heat, cooled to room temperature, and acidified to pH 2.8-3 with 36% HCl (11.6 M). The mixture was refrigerated at 10° C. for an hour. The solid-liquid solution was removed from the fridge and filtered to recover a white precipitate. The resulting solid mass of 24 grams contained 22 grams (S,S)-EDDS and 2 grams aspartic acid, representing a 50% yield of (S,S)-EDDS at 92% purity. The aqueous liquid filtrate was removed, acidified further to pH 2.5 and refrigerated at 10° C. for 1 h. The cooled solution was filtered again to yield 11 grams of aspartic acid and 4 g of EDDS. The liquid filtrate was placed in the fridge for 24 hours which resulted in more white solid formation. The white solid was filtered resulting in 5 grams which contained 1.4 grams of EDDS and 3.6 grams of aspartic acid.

### Example 2

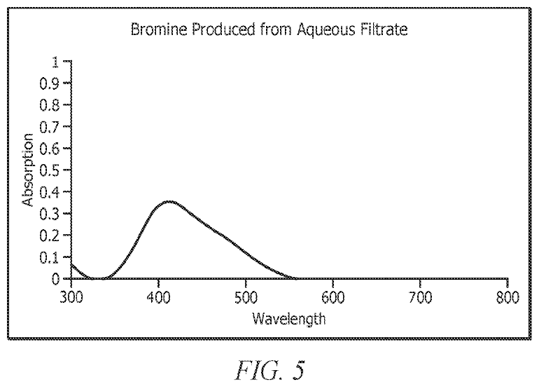

In a second example, the aqueous filtrate from example 1 was poured into a 3-neck round bottom flask attached to a distillation condenser, heating and stirring apparatus, and an addition funnel. 4.3 mL of 36% HCl (11.6 M) and 28 mL of 6% Sodium hypochlorite were added respectively via an addition funnel into the flask containing the filtrate. Bromine gas was driven off and collected as a liquid in a condenser. UV-vis of the prepared bromine upon dilution in dichloromethane is shown in FIG. 5. Preparation of DBE from the produced bromine and ethylene can be carried out in accordance with the commercial process in a glass-column reactor with coil heat exchangers at 100° C. in accordance with known processes.

### Example 3

In a third example, generation of molecular chlorine (Cl₂) in water and its reaction with spent bromide from the EDDS reaction was carried out. Concentrated HCl (35%) and hypochlorite (sodium or calcium salt) were added respectively to a 3-neck round bottom flask attached to a distillation condenser, heating and stirring apparatus, and an addition funnel. To the generated molecular chlorine, the aqueous filtrate from example 1 (EDDS synthesis) was added to via an addition funnel to generate bromine, which was collected through a condenser in flask for reaction with ethylene.

### Example 4

In a fourth example, molecular chlorine (Cl₂) gas was bubbled through the aqueous filtrate from example 1 to oxidize spent bromide to bromine (Bra), which can be removed, condensed and reacted with ethylene to make DBE. This example demonstrates that alternative oxidants can be used to regenerate the bromine in the aqueous filtrate.

Having described various systems, reactors, and processes, certain aspects can include, but are not limited to:

In a first aspect, a process for synthesizing EDDS comprises: combing an aqueous solution of aspartic acid with dibromoethane under basic conditions to form a reaction solution; heating the reaction solution to form a heated reaction solution, wherein the heated reaction solution comprises a reaction product, wherein the reaction product comprises (S,S) ethylenediamine-N,N′-disuccinic acid (EDDS); and recovering at least a portion of the reaction product from the reaction solution.

A second aspect can include the process of the first aspect, wherein recovering at least the portion of the reaction product comprises: cooling the heated reaction solution to form a cooled reaction solution; and precipitating at least a portion of the reaction product from the cooled reaction solution.

A third aspect can include the process of the first or second aspect, wherein the reaction product comprises the EDDS at a purity of at least about 40%.

A fourth aspect can include the process of any one of the first to third aspects, wherein the aspartic acid comprises L-aspartic acid.

A fifth aspect can include the process of any one of the first to fourth aspects, wherein the aqueous solution has a pH of greater than 9.

A sixth aspect can include the process of any one of the first to fifth aspects, wherein heated reaction solution has a temperature between about 50° C. and about 100° C.

A seventh aspect can include the process of any one of the first to sixth aspects, further comprising: continuously stirring the reaction solution and the heated reaction solution.

An eighth aspect can include the process of any one of the first to seventh aspects, where the dibromoethane is combined with the aqueous solution over a period of about thirty minutes to about 5 hours.

A ninth aspect can include the process of any one of the first to seventh aspects, wherein the dibromoethane is combined with the aqueous solution as a single dose.

A tenth aspect can include the process of any one of the first to ninth aspects, further comprising: maintaining the heated reaction solution at a pH between about 9-14, or between about 9 and about 10.

An eleventh aspect can include the process of the tenth aspect, wherein the heated reaction solution is maintained at the pH using periodic or continuous addition of base to the heated reaction solution.

A twelfth aspect can include the process of any one of the first to eleventh aspects, wherein the cooled reaction solution is at about room temperature.

A thirteenth aspect can include the process of any one of the second to twelfth aspects, wherein precipitating at least the portion of the reaction product comprises acidifying the cooled reaction solution.

A fourteenth aspect can include the process of the thirteenth aspect, wherein the cooled reaction solution is acidified to a pH of between about 2-4, or between about 2.8 and about 3.

A fifteenth aspect can include the process of the thirteenth or fourteenth aspect, wherein the cooled reaction solution is acidified using hydrochloric acid (HCl).

A sixteenth aspect can include the process of any one of the first to fifteenth aspects, wherein recovering at least the portion of the reaction product from the reaction solution forms a liquid filtrate, wherein the process further comprises: acidifying the liquid filtrate; forming a precipitate in response to the acidifying, wherein the precipitate comprises unreacted aspartic acid.

A seventeenth aspect can include the process of the sixteenth aspect, further comprising: combining the liquid filtrate with sodium hypochlorite and hydrochloric acid; forming bromine gas in response to the combining.

An eighteenth aspect can include the process of the sixteenth or seventeenth aspect, further comprising: combining sodium hypochlorite and hydrochloric acid to produce molecular chlorine (Cl₂) in water; combining the chlorine in the water with the liquid filtrate; and forming bromine gas in response to the combining.

A nineteenth aspect can include the process of any one of the sixteenth to eighteenth aspects, further comprising: bubbling gaseous chlorine directly for reaction with the aqueous liquid filtrate forming bromine.

A twentieth aspect can include the process of the eighteenth aspect, wherein the chlorine is present below the solubility limit of the chlorine in the water.

A twenty first aspect can include the process of the sixteenth aspect, further comprising: passing chlorine gas through an aqueous solution; dissolving at least a portion of the chlorine gas in the aqueous solution; combining the chlorine dissolved in the aqueous solution with the liquid filtrate; and forming bromine gas in response to the combining.

A twenty second aspect can include the process of any one of the first to twenty first aspects, further comprising: combining the bromine gas with ethylene to form dibromoethane.

A twenty third aspect can include the process of the twenty second aspect, further comprising: recycling the dibromoethane to combine with the aqueous solution.

In a twenty fourth aspect, a process for synthesizing (S,S) EDDS from L-aspartic acid and dibromoethane comprises: combing an aqueous solution of L-aspartic acid with dibromoethane under basic conditions to form a reaction solution; heating the reaction solution to form a heated reaction solution, wherein the heated reaction solution comprises a reaction product, wherein the reaction product comprises (S,S) ethylenediamine-N,N′-disuccinic acid (EDDS); cooling the heated reaction solution to form a cooled reaction solution; precipitating at least a portion of the reaction product from the cooled reaction solution; and recovering the portion of the reaction product from the cooled reaction solution, wherein the reaction product comprises ≥40% pure (S,S)-EDDS.

In a twenty fifth aspect, a process for synthesizing (S,S)-EDDS from L-aspartic acid and dibromoethane comprises: combing an aqueous solution of L-aspartic acid with dibromoethane under basic conditions to form a reaction solution; forming a reaction product based on the combining, wherein the reaction product comprises (S,S) ethylenediamine-N,N′-disuccinic acid (EDDS); and separating the EDDS as a solid product from the reaction mixture after the forming.

In a twenty sixth aspect, a method comprises producing ≥40% pure (S,S)-EDDS or ≥90% pure (S,S)-EDDS stoichiometrically from aspartic acid, ethylene and hypochlorite using bromine as a carrier.

A twenty seventh aspect can include the method of the twenty sixth aspect, wherein the method comprises the recovery of unreacted L-aspartic acid.

A twenty eighth aspect can include the method of the twenty sixth or twenty seventh aspect, further comprising producing bromine from spent bromide filtrate using bleach, molecular chlorine, hydrogen peroxide, organic peroxide or other oxidant, or electrocatalysis.

A twenty ninth aspect can include the method of any one of the twenty sixth to twenty eighth aspects, further comprising generating dibromoethane from reaction of bromine and ethylene.

While several embodiments have been provided in the present disclosure, it should be understood that the disclosed systems and methods may be embodied in many other specific forms without departing from the spirit or scope of the present disclosure. The embodiments and present examples are to be considered as illustrative and not restrictive, and the intention is not to be limited to the details given herein. Many variations and modifications of the systems and methods disclosed herein are possible and are within the scope of the disclosure. For example, the various elements or components may be combined or integrated in another system or certain features may be omitted or not implemented. Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

Numerous other modifications, equivalents, and alternatives, will become apparent to those skilled in the art once the above disclosure is fully appreciated. It is intended that the following claims be interpreted to embrace all such modifications, equivalents, and alternatives where applicable. Accordingly, the scope of protection is not limited by the description set out above but is only limited by the claims which follow, that scope including all equivalents of the subject matter of the claims. Each and every claim is incorporated into the specification as an embodiment of the present systems and methods. Thus, the claims are a further description and are an addition to the detailed description of the present invention. The disclosures of all patents, patent applications, and publications cited herein are hereby incorporated by reference.

1. A process for synthesizing EDDS, the process comprising:
combining an aqueous solution of aspartic acid with dibromoethane under basic conditions to form a reaction solution;
heating the reaction solution to form a heated reaction solution, wherein the heated reaction solution comprises a reaction product, wherein the reaction product comprises (S,S) ethylenediamine-N,N′-disuccinic acid (EDDS); and
recovering at least a portion of the reaction product from the reaction solution.

2. The process of claim 1, wherein recovering at least the portion of the reaction product comprises:
cooling the heated reaction solution to form a cooled reaction solution; and
precipitating at least a portion of the reaction product from the cooled reaction solution.

3. The process of claim 1, wherein the reaction product comprises the EDDS at a purity of at least about 40%.

4. The process of claim 1, wherein the aspartic acid comprises L-aspartic acid, wherein the aqueous solution has a pH of greater than 9, and wherein the heated reaction solution has a temperature between about 50° C. and about 100° C..

5. (canceled)

6. (canceled)

7. The process of claim 1, further comprising: continuously stirring the reaction solution and the heated reaction solution.

8. The process of claim 1, where the dibromoethane is combined with the aqueous solution over a period of about thirty minutes to about 5 hours, or wherein the dibromoethane is combined with the aqueous solution as a single dose.

9. (canceled)

10. The process of claim 1, further comprising: maintaining the heated reaction solution at a pH between about 9-14 using periodic or continuous addition of base to the heated reaction solution.

11. (canceled)

12. The process of claim 1, wherein the cooled reaction solution is at about room temperature.

13. The process of claim 2, wherein precipitating at least the portion of the reaction product comprises acidifying the cooled reaction solution to a pH of between about 2-4.

14. (canceled)

15. The process of claim 13, wherein the cooled reaction solution is acidified using hydrochloric acid (HCl).

16. The process of claim 1, wherein recovering at least the portion of the reaction product from the reaction solution forms a liquid filtrate, wherein the process further comprises:
acidifying the liquid filtrate;
forming a precipitate in response to the acidifying, wherein the precipitate comprises unreacted aspartic acid.

17. The process of claim 16, further comprising:
combining the liquid filtrate with sodium hypochlorite and hydrochloric acid;
forming bromine gas in response to the combining.

18. The process of claim 16, further comprising:
combining sodium hypochlorite and hydrochloric acid to produce molecular chlorine (Cl₂) in water;
combining the chlorine in the water with the liquid filtrate; and
forming bromine gas in response to the combining.

19. The process of claim 16, further comprising:
bubbling gaseous chlorine directly for reaction with the aqueous liquid filtrate forming bromine.

20. The process of claim 19, wherein the chlorine is present below the solubility limit of the chlorine in the water.

21. The process of claim 16, further comprising:
passing chlorine gas through an aqueous solution;
dissolving at least a portion of the chlorine gas in the aqueous solution;
combining the chlorine dissolved in the aqueous solution with the liquid filtrate; and
forming bromine gas in response to the combining.

22. The process of claim 1, further comprising:
combining the bromine gas with ethylene to form dibromoethane.

23. The process of claim 22, further comprising:
recycling the dibromoethane to combine with the aqueous solution.

24. A process for synthesizing (S,S) EDDS from L-aspartic acid and dibromoethane, the process comprising:
combining an aqueous solution of L-aspartic acid with dibromoethane under basic conditions to form a reaction solution;
heating the reaction solution to form a heated reaction solution, wherein the heated reaction solution comprises a reaction product, wherein the reaction product comprises (S,S) ethylenediamine-N,N′-disuccinic acid (EDDS);
cooling the heated reaction solution to form a cooled reaction solution;
precipitating at least a portion of the reaction product from the cooled reaction solution; and
recovering the portion of the reaction product from the cooled reaction solution, wherein the reaction product comprises ≥40% pure (S,S)-EDDS.

25. A process for synthesizing (S,S)-EDDS from L-aspartic acid and dibromoethane, the process comprising:
combining an aqueous solution of L-aspartic acid with dibromoethane under basic conditions to form a reaction solution;
forming a reaction product based on the combining, wherein the reaction product comprises (S,S) ethylenediamine-N,N′-disuccinic acid (EDDS); and
separating the EDDS as a solid product from the reaction mixture after the forming.
